(19) United States
(12) Patent Application Publication
(10) Pub. No.: US 2001/0047097 A1
(43) Pub. Date: Nov. 29, 2001

(54) Process for the preparation of arylethylamines by amination of arylolefins

(76) Inventors: Harald Trauthwein, Munchen (DE); Matthais Beller, Rostock (DE); Claudia Breindl, Munchen (DE); Christian Hartung, Munchen (DE); Annegret Tillack, Rostock (DE)

Correspondence Address: Ashley I. Pezzner, Esquire, CONNOLLY BOVE LODGE & HUTZ LLP, 1220 Market Street, P.O. Box 2207, Wilmington, DE, 19899, US

(21) Appl. No.: 09/859,265

(22) Filed: May 17, 2001

(30) Foreign Application Priority Data
May 20, 2000 (DE) 100 25 114.5

(51) Int. Cl. C07D 265/30; C07D 211/08

(52) U.S. Cl. 544/178; 546/192; 564/373

(57) ABSTRACT

The invention relates to a process for the preparation of 2-arylethylamine derivates having an alkyl substituent in the 1-position, of the formula (I)

aryl-CHR¹—CR²(NR³R⁴)—(CHR⁵R⁶)ₙ—CHR⁷R⁸ (I)

by reaction of aromatic olefins of the general formulae (Ila-c)

aryl-CHR¹—CHR²—(CR⁵R⁶)ₐ—CR⁵═CR⁶—(CR⁵R⁶)_{b}CHR⁷R⁸ (IIa)

aryl-CHR¹—CR²═CR⁵—(CR⁵R⁶)_{c}—CHR⁷R⁸ (IIb)

aryl-CHR¹—CHR²—(CR⁵CR⁶)_{c}—CR⁵═CR⁷CR⁸ (IIc)

with amines of the general formula (III)

R³R⁴NH

in the presence of a base.

### DESCRIPTION

The present invention relates to a process for the preparation of 2-arylethylamines from arylolefins and amines in the presence of a base as catalyst.

2-Arylethylamine derivatives having an alkyl substituent in the 1-position (aryl-CHR¹—CHR²—NR³R⁴, amphetamines) are an important class of compounds in the field of pharmaceutical chemistry. Depending on substituents, 2-arylethylamines exhibit various biological actions and have industrial importance as pharmaceuticals in a number of indication areas. Examples of amphetamines employed pharmaceutically are fenfluramine (appetite suppressant), prolintane (sympathomimetic), fenetylline (sympathomimetic) and bufotenine (psychodysleptic).

Generally, 2-arylethylamines and their derivatives are prepared by reaction (nucleophilic substitution) of 2-arylethyl halides with amines. In this process, at least stoichiometric amounts of by-products (salt wastes) are formed. In addition, the yields of the corresponding nucleophilic substitutions are not good, as multiple alkylations occur. A further synthesis of 2-arylethylamine derivatives starts from arylacetaldehyde derivatives, which are reductively aminated in the presence of a transition metal catalyst (see, for example (a) Glennon, R. A.; Smith, J. D.; Ismaiel, A. M.; El-Ashmawy, M.; Battaglia, G.; Fisher, J. B. J. Med. Chem. 1991, 34, 1094; (b) Nicols, D. E.J. Med. Chem. 1973, 16, 480). Problems of this process are the accessibility of the arylacetaldehyde derivatives and the costs of the transition metal catalyst.

A process which avoids the disadvantages of the abovementioned laboratory syntheses is the base-catalyzed amination of styrenes. Here, amines are added to styrenes in the presence of a base in an atom-efficient manner. Examples of this reaction are found in Beller, M.; Breindl, C. Tetrahedron 1998, 54, 6359. This process is problematic, however, when amphetamines, i.e. 2-arylethylamines having a further alkyl substituent in the 1-position, are to be synthesized, as the necessary starting compounds cannot be prepared in a simple and practicable manner.

For the reasons mentioned, a need existed for a novel process which makes 2-arylethylamine derivatives having an alkyl substituent in the 1-position accessible in a simple manner from inexpensive, readily obtainable starting materials, and which does not have the disadvantages of the known preparation processes and is thus suitable for carrying out industrially and yields the 2-arylethylamine derivatives having an alkyl substituent in the 1-position in high yield, catalyst productivity and purity.

It was surprisingly found that in the presence of bases 2-arylethylamine derivatives having an alkyl substituent in the 1-position are obtainable from arylolefins of the formulae (IIa-c) and amines. The amination in this case takes place with high selectivity in the 2-position of the arylolefin, almost independently of the position of the double bond in the olefinic radical.

The more readily accessible arylolefins which have a double bond in the 2-position or higher in the olefinic radical can thus be used for the preparation of 2-aryiethylamine derivatives having an alkyl substituent in the 1-position. The specific amination in the 2-position of the arylolefin appears to precede an isomerization reaction (domino isomerization). Thus in the synthesis of a specific 2-arylethylamine derivative isomer mixtures of an arylolefin with a different position of the double bond can also be directly employed.

The invention consequently relates to a process for the preparation of 2-arylethylamine derivatives of the formula (I)

aryl-CHR¹—CR²(NR³R⁴)—(CHR⁵R⁶)ₙ—CHR⁷R⁸ (I)

in which R¹ to R⁶

can be, identically or differently, hydrogen, C₁-C₂₄-alkyl, C₃-C₁₂-cycloalkyl, where the cycle can also contain 1-2 heteroatoms selected from the group consisting of N, O, S, P and the ring size is 3-12, phenyl, naphthyl, fluorenyl, C₆-C₁₄-aryl, C₂-C₁₃-heteroaryl, where the number of heteroatoms selected from the group consisting of N, O, S, P can be 1 to 4 and the ring size is 3-14,

where these groups themselves can in each case be mono- or polysubstituted and these substituents in this case independently of one another contain hydrogen, C₁-C₂₀-alkyl, C₁-C₁₀-fluoroalkyl, C₃-C₁₂-cycloalkyl, where the cycle can also contain 1-2 heteroatoms selected from the group consisting of N, O, S, P and the ring size is 3-12, phenyl, C₆-C₁₄-aryl, C₃-C₁₃-heteroaryl, where the number of heteroatoms selected from the group consisting of N, O, S, P can be 1 to 4 and the ring size is 3-14, C₁-C₁₀-alkoxy, C₆-C₁₄-aryloxy, C₃-C₁₃-heteroaryloxy, where the number of heteroatoms selected from the group consisting of N, O, S, P can be 1 to 4 and the ring size is 3-14, C₁-C₉-trifluoromethylalkyl, trifluoromethyl, fluoro, nitro, hydroxyl, trifluoromethylsulfonato, thio, thiolato, C₁-C₈-substituted amino of the forms NH-alkyl-C₁-C₈, NH-aryl-C₅-C₆, N-alkyl₂-C₁-C₈, N-aryl₂-C₅-C₆, NHCOH, NH—CO-alkyl-C₁-C₈, NH—CO-aryl-C₅-C₆, NHCOO-alkyl-(C₁-C₄); NHCOO-aryl-(C₃-C₈); cyano, C₁-C₆-acyloxy, C₁-C₇-CO₂H, SO₂-aryl-(C₃-C₆), SO-aryl-(C₃-C₆), SO₂-alkyl-(C₁-C₆), SO-alkyl-(C₁-C₆), sulfinato, sulfonato of the forms SO₃H and SO₃R⁹, P(phenyl)₂, P-alkyl₂-(C₁-C₈), P-aryl₂-(C₃-C₈), PO-phenyl₂, POalkyl₂-(C₁-C₄), phosphato of the forms PO₃H₂, PO₃HR⁹, PO₃ R⁹₂, CONH₂, CONR⁹₂, CONHR⁹, where R⁹ is either a C₁-C₈-alkyl or C₆-aryl, C₁-C₆-trialkylsilyl,

where, for clarification, it is pointed out that even the individual substituents R⁵ and R⁶ in the n subunits are to be regarded as substituents which are independent of one another, and

in which R⁷and R⁸

can be, identically or differently, hydrogen, C₁-C₂₄-alkyl, C₃-C₁₂-cycloalkyl, where the cycle can also contain 1-2 heteroatoms selected from the group consisting of N, O, S, P and the ring size is 3-12, phenyl, naphthyl, fluorenyl, C₆-C₁₄-aryl, C₂-C₁₃-heteroaryl, where the number of heteroatoms selected from the group consisting of N, O, S, P can be 1 to 4 and the ring size is 3-14, fluorine, OH, NO₂, CN, O—C₁-C₂₄-alkyl, O—C₃-C₁₂-cycloalkyl, where the cycle can also contain 1-2 heteroatoms selected from the group consisting of N, O, S, P and the ring size is 3-12,

O—C₆-C₁₄-aryl, O—C₂-C₁₃-heteroaryl, where the number of heteroatoms selected from the group consisting of N, O, S, P can be 1 to 4 and the ring size is 3-14,

N—(C₁-C₂₄)₂-alkyl, N-(C₃-C₈)₂-cycloalkyl, where the cycle can also contain 1-2 heteroatoms selected from the group consisting of N, O, S, P and the ring size is 3-12,

N—(C₆-C₁₄-aryl)₂, N—(C₂-C₁₃-heteroaryl)₂, where the number of heteroatoms selected from the group consisting of N, O, S, P can be 1 to 4 and the ring size is 3-14,

O₂C—C₁-C₂₄-alkyl, O₂C—C₃-C₈-cycloalkyl where the cycle can also contain 1-2 heteroatoms selected from the group consisting of N, O, S, P and the ring size is 3-12, O₂C—C₆-C₁₄-aryl,

NR¹⁰—CO—(C₁-C₂₄)₂-alkyl, NR¹⁰—CO—(C₃-C₁₂)₂-cycloalkyl, where the cycle can also contain 1-2 heteroatoms selected from the group consisting of N, O, S, P and the ring size is 3-12,

NR¹⁰—CO—(C₆-C₁₄-aryl)₂, NR¹⁰—CO—(C₂-C₁₃-heteroaryl)₂, where the number of heteroatoms selected from the group consisting of N, O, S, P can be 1 to 4 and the ring size is 3-14, where R¹⁰ is C₁-C₈-alkyl or C₆-aryl

Si—(C₁-C₂₄)₄-alkyl, Si—(C₃-C₁₂)₄-cycloalkyl, where the cycle can also contain 1-2 heteroatoms selected from the group consisting of N, O, S, P and the ring size is 3-12,

Si—(C₆-C₁₄-aryl)₄, Si—(C₂-C₁₃-heteroaryl)₄, where the number of heteroatoms selected from the group consisting of N, O, S, P can be 1 to 4 and the ring size is preferably 3-14,

where these groups themselves can in each case be mono- or polysubstituted and these substituents in this case independently of one another can be hydrogen, C₁-C₂₀-alkyl, C₁-C₁₀-fluoroalkyl, C₃-C₁₂-cycloalkyl, where the cycle can also contain 1-2 heteroatoms selected from the group consisting of N, O, S, P and the ring size is 3-12, phenyl, C₆-C₁₄-aryl, C₃-C₁₃-heteroaryl, where the number of heteroatoms selected from the group consisting of N, O, S, P can be 1 to 4 and the ring size is 3-14, C₁-C₁₀-alkoxy, C₆-C₁₄-aryloxy, C₃-C₁₃-heteroaryloxy, where the number of heteroatoms selected from the group consisting of N, O, S, P can be 1 to 4 and the ring size is 3-14, C₁-C₉-trifluoromethylalkyl, trifluoromethyl, fluoro, nitro, hydroxyl, trifluoromethylsulfonato, thio, thiolato, C₁-C₈-substituted amino of the forms NH-alkyl-C₁-C₈, NH-aryl-C₅-C₆, N-alkyl₂-C₁-C₈, N-aryl₂-C₅-C₆, NHCOH, NH—CO-alkyl-C₁-C₈, NH—CO-aryl-C₅-C₆, NHCOO-alkyl-(C₁-C₄); NHCOO-aryl-(C₃-C₈); cyano, C₁-C₆-acyloxy, C₁-C₇-CO₂H, SO₂-aryl-(C₃-C₆), SO-aryl-(C₃-C₆), SO₂-alkyl-(C₁-C₆), SO-alkyl-(C₁-C₆), sulfinato, sulfonato of the forms SO₃H and SO₃R⁹, P(phenyl)₂, P-alkyl₂-(C₁-C₈), P-aryl₂-(C₃-C₈), PO-phenyl₂, POalkyl₂-(C₁-C₄), phosphato of the forms PO₃H₂, PO₃HR⁹, PO₃ R⁹₂, CONH₂, CONR⁹₂, CONHR⁹, where R⁹ is either a C₁-C₈-alkyl or C₆-aryl, C₁-C₆-trialkylsilyl,

and in which aryl in formula (I)

can be C₃-C₁₄-aryl, C₂-C₁₃-heteroaryl, where the number of heteroatoms selected from the group consisting of N, O, S, P can be 1 to 4 and the ring size is 3-14,

where these groups themselves can in each case be mono- or polysubstituted and these substituents in this case independently of one another can be hydrogen, C₁-C₂₀-alkyl, C₁-C₁₀-fluoroalkyl, C₃-C₁₂-cycloalkyl, where the cycle can also contain 1-2 heteroatoms selected from the group consisting of N, O, S, P and the ring size is 3-12, phenyl, C₆-C₁₄-aryl, C₂-C₁₃-heteroaryl, where the number of heteroatoms selected from the group consisting of N, O, S, P can be 1 to 4 and the ring size is 3-14, C₁-C₁₀-alkoxy, C₆-C₁₄-aryloxy, C₁-C₉-trifluoromethylalkyl, trifluoromethyl, fluoro, nitro, hydroxyl, trifluoromethylsulfonato, thio, thiolato, C₁-C₈-substituted amino of the forms NH-alkyl-C₁-C₈, NH-aryl-C₅-C₆, N-alkyl₂-C₁-C₈, N-aryl₂-C₅-C₆, N-alkyl₃-C₁-C₈⁺, N-aryl₃-C₅-C₆⁺, NHCOH, NH—CO-alkyl-C₁-C₈, NH—CO-aryl-C₅-C₆, NHCOO-alkyl-(C₁-C₄); NHCOO-aryl-(C₃-C₈); cyano, C₁-C₆-acyloxy, SO₂-aryl-(C₃-C₆), SO-aryl-(C₃-C₆) SO₂-alkyl-(C₁-C₆), SO-alkyl-(C₁-C₆), sulfinato, sulfonato of the forms SO₃H and SO₃R⁹, P(phenyl)₂, CHCHCO₂H, P-alkyl₂-(C₁-C₈), P-aryl₂-(C₃-C₈), PO-phenyl₂, POalkyl₂-(C₁-C₄), phosphato of the forms PO₃H₂, PO₃HR⁹ and PO₃R⁹₂, CONH₂, CONR⁹₂, CONHR⁹ where R⁹ is either C₁-C₈-alkyl or C₆-aryl, C₁-C₆-trialkylsilyl, and

in which n is a number between 0 and 11,

by reaction of aromatic olefins of the general formulae (IIa-c)

aryl-CHR¹—CHR²—(CR⁵R⁶)ₐ—CR⁵═CR⁶—(CR⁵R⁶)_{b}—CH R⁷R⁸ (IIa)

aryl-CHR¹—CR²═CR⁵—(CR⁵R⁶)_{c}—CHR⁷R⁸ (IIb)

aryl-CHR¹—CHR²—(CR⁵CR⁶)_{c}—CR⁵═CR⁷CR⁸ (IIc)

with amines of the general formula (III),

R³R⁴NH (III)

in which in the formulae of the type II and III R¹ to R⁸ have the meaning indicated beforehand for the formulae of the type (I) and a and b are a number between 0-9 with the condition that a+b is <10 and c is a number between 0-10,

in the presence of a base, in particular of a Brönsted base. Preferred bases are alkali metal and/or alkaline earth metals (e.g. sodium, lithium, potassium, calcium), alkali metal and/or alkaline earth metal hydrides (e.g. sodium hydride, lithium hydride, magnesium hydride, calcium hydride), alkali metal and/or alkaline earth metal amides (e.g. lithium diisopropylamide, sodium amide, lithium diethylamide, sodium dimethylamide), alkali metal and/or alkaline earth metal alkoxides (e.g. potassium tert-butoxide) and alkali metal and/or alkaline earth metal hydrocarbons (e.g. butyllithium, methyllithium, phenyllithium, phenylsodium, diethylmagnesium). Alkali metals, alkali metal and/or alkaline earth metal hydrides, alkali metal amides and alkali metal hydrocarbons are particularly preferred.

A process is particularly preferred in which compounds of the formula (I) are prepared in which R¹ to R⁶

can be hydrogen, C₁-C₈-alkyl, C₆-C₁₄-aryl, C₃-C₁₂-cycloalkyl, where the cycle can also contain 1-2 heteroatoms selected from the group consisting of N, O, S, P and the ring size is 3-12,

where these groups themselves can in each case be mono- to trisubstituted and these substituents in this case independently of one another can be hydrogen, C₁-C₂₀-alkyl, C₆-C₁₄-aryl, C₃-C₁₂-cycloalkyl, where the cycle can also contain 1-2 heteroatoms selected from the group consisting of N, O, S, P and the ring size is 3-12, C₁-C₁₀-alkoxy, C₁-C₁₄-aryloxy, C₃-C₁₃-heteroaryloxy, where the number of heteroatoms selected from the group consisting of N, O, S, P can be 1 to 4 and the ring size is 3-14, fluoro, trifluoromethyl, N-alkyl₂-C₁-C₈, N-aryl₂-C₅-C₆

and in which R⁷ and R⁸ independently of one another can be hydrogen, fluoro, trifluoromethyl, C₆-C₁₄-aryl, O—C₁-C₈-alkyl, O—C₃-C₁₂-cycloalkyl, where the cycle can also contain 1-2 heteroatoms selected from the group consisting of N, O, S, P and the ring size is 3-12, O—C₆-C₁₄-aryl, O—C₂-C₁₃-heteroaryl, where the number of heteroatoms selected from the group consisting of N, O, S, P can be 1 to 4 and the ring size is preferably 3-14,

N—(C₁-C₈)₂-alkyl, N—(C₃-C₈)₂-cycloalkyl, where the cycle can also contain 1-2 heteroatoms selected from the group consisting of N, O, S, P and the ring size is 3-12,

N—(C₆-C₁₄-aryl)₂, N—(C₂-C₁₃-heteroaryl)₂, where the number of heteroatoms selected from the group consisting of N, O, S, P can be 1 to 4 and the ring size is 3-14,

where these groups themselves in each case can be mono- to trisubstituted and these substituents in this case independently of one another can be hydrogen, C₁-C₂₀-alkyl, C₃-C₁₂-cycloalkyl, where the cycle can also contain 1-2 heteroatoms selected from the group consisting of N, O, S, P and the ring size is 3-12, phenyl, C₆-C₁₄-aryl, C₃-C₁₃-heteroaryl, where the number of heteroatoms selected from the group consisting of N, O, S, P can be 1 to 4 and the ring size is 3-14, C₁-C₁₀-alkoxy, C₆-C₁₄-aryloxy, C₃-C₁₃-heteroaryloxy, where the number of heteroatoms selected from the group consisting of N, O, S, P can be 1 to 4 and the ring size is 3-14, N-alkyl₂-C₁-C₈, N-aryl₂-C₅-C₆, trifluoromethyl, fluoro.

Particularly preferred substituents are also phenyl, naphthalene, phenanthrene, pyrrole, furan, thiophene, indole, quinoline, benzofuran

and in which aryl in formula (I)

is C₃-C₁₄-aryl, C₂-C₁₃-heteroaryl, where the number of heteroatoms selected from the group consisting of N, O, S, P can be 1 to 4 and the ring size is 3-14,

where these groups themselves can in each case be mono- to trisubstituted and these substituents in this case independently of one another can be hydrogen, C₁-C₂₀-alkyl, C₃-C₁₂-cycloalkyl, where the cycle can also contain 1-2 heteroatoms selected from the group consisting of N, O, S, P and the ring size is 3-12, phenyl, C₆-C₁₄-aryl, C₂-C₁₃-heteroaryl, where the number of heteroatoms selected from the group consisting of N, O, S, P can be 1 to 4 and the ring size is 3-14, C₁-C₁₀-alkoxy, C₁-C₉-trifluoromethylalkyl, trifluoromethyl, fluoro, nitro, hydroxyl, trifluoromethylsulfonato, thio, thiolato

and in which n is a number between 0 and 7.

The preferred ring size is of the cycloalkyl, heterocycloalkyl, aryl and heteroaryl substituents are 5 to 7.

The process according to the invention has proven very particularly suitable for the preparation of amphetamines in which R¹ to R⁶ independently of one another are hydrogen, C₁-C₈-alkyl, C₆-C₁₄-aryl, C₃-C₁₂-cycloalkyl, where the cycle can also contain 1-2 heteroatoms selected from the group consisting of N, O, S, P and the ring size is 3-12,

and in which R⁷ and R⁸ are hydrogen, C₃-C₁₄-aryl, C₂-C₁₃-heteroaryl, where the number of heteroatoms selected from the group consisting of N, O, S, P can be 1 to 4 and the ring size is 3-14,

and in which n is a number between 0 and 7.

In the context of the process according to the invention, olefin mixtures of the formula II can also be advantageously employed, arylethylamines of the formula I selectively being obtained as products. Olefin mixtures of the formula II can be prepared, for example, by Heck reaction of aryl halides using inexpensive olefins (see M. Beller, T. H. Riermeier, G. Stark in Transition Metals for Organic Synthesis (Eds. M. Beller, C. Bolm) Vol. I, pp. 208-240, Wiley-VCH, Weinheim, 1998).

Solvents used in the process are in general inert organic solvents. Aliphatic and aromatic ethers (MTBE, THF, dioxane, anisole, diethyl ether, dibutyl ether, etc.), polyethers (polyethylene glycols, etc.), aromatic and/or aliphatic hydrocarbons (toluene, xylene, tetralin, octane, etc.) and their mixtures are particularly suitable. In addition, the reaction can also be carried out in tert. amines (triethylamine, tributylamine, methyl diisopropylamine, etc.), dipolar aprotic solvents (DMSO, DMAC, NMP, tetramethylurea, etc.) or without solvent.

The reaction proceeds at temperatures from −70 to 200° C.; in many cases it has proven suitable to work at temperatures of 0 to 180° C., preferably 20 to 140° C. The reaction can be carried out under pressure, in particular if low-boiling amines are employed.

A base must be added to the reaction mixture as catalyst. The basic catalyst serves to deprotonate the amine present to the corresponding amide. Both the base and the corresponding amide catalyze the isomerization of the double bond and the subsequent amination. Suitable bases are those compounds which are able to deprotonate the amine in low concentration. Particularly suitable for this purpose are alkali metal and/or alkaline earth metal alkoxides such as KOtBu, alkali metal and/or Ah alkaline earth metal amides, alkali metal and/or alkaline earth metal elements and/or gFEj alkali metal and/or alkaline earth metal hydrocarbons such as butyllithium, phenyllithium and/or hydroxides, preferably of lithium, sodium, potassium, calcium, magnesium, cesium. ‘Solid superbases’ (e.g. alkali metal-doped zeolites) can also be employed as catalysts.

In the process according to the invention, the base is preferably employed in catalytic amounts relative to the amine. Amounts of catalyst from 0.01 eq to 0.5 eq are preferably used. 0.05 eq-0.4 eq of base catalyst is particularly preferably employed.

In some cases, it has turned out to be positive to add a cocatalyst stabilizing the intermediately formed alkali metal or alkaline earth metal amide. Cocatalysts which can be employed are, in particular, chelating diamines such as TMEDA but also trialkylamines or ethers, which can be alicyclic and/or open-chain. The cocatalyst is used in amounts from 0.01 eq to 1.5 eq (based on amine). 0.05 eq-1 eq of cocatalyst is preferably employed.

The amines which can be prepared according to the invention are of great importance as pharmaceuticals.

### EXAMPLES

The examples below serve to illustrate the process according to the invention without restricting it thereto.

General Working Procedure

The amine (2.5 mmol) and 100 μl of hexadecane (internal GC standard) are dissolved in 5 ml of absolute tetrahydrofuran in an Aldrich Ace pressure tube under an argon protective gas atmosphere. 20 mol % of n-butyllithium (1.6M of n-BuLi solution in hexane) are slowly added at room temperature. The solution is stirred for 10 minutes before adding allylbenzene (5 mmol). The usually intensively colored solution is reacted at the temperature indicated for 20 hours. After cooling, the mixture is hydrolyzed using 2 ml of water, decoloration of the solution occurring. For the isolation of the product, the batch is treated with 5 ml of 1M hydrochloric acid and 5 ml of dichloromethane. The aqueous phase is separated off and the organic phase is extracted three times with 5 ml of 1M hydrochloric acid in each case. The combined aqueous phases are neutralized with Na₂CO₃ and extracted five times with 5 ml of dichloromethane in each case. The organic phases are washed with water and dried over MgSO₄. After removing the solvent in vacuo, the product is isolated by means of column chromatography.

### Example 1

N-2-(1-Phenyl)propylpiperidine. Piperidine (2.5 mmol; 247 μl) and allylbenzene (5 mmol; 662 μl) are reacted at room temperature according to the general working procedure with addition of 20 mol % of n-BuLi solution (0.5 mmol; 313 μl). The product is purified by column chromatography (n-hexane/ethyl acetate=3:1). N-2-(1-Phenyl)propylpiperidine is obtained as a colorless oil—yield: 89% (GC); 84% (isolated).—¹H NMR (CDCl₃, 400.1 MHz, 25° C., δ=ppm): 7.24 (m, 2H, phenyl); 7.15 (m, 3H, phenyl); 3.00 (dd, ²J(H, H)=12.9 Hz, ³J(H, H)=3.8 Hz, 1H, Ph-CH₂); 2.76 (ddq, ³J(H, H)=10.1 Hz, ³J(H, H)=6.5 Hz, ³J(H, H)=3.8 Hz, 1H, Ph-GH₂—CH); 2.54 (m, 4H, N—CH₂); 2.36 (dd, ²J(H, H)=12.9 Hz, ³J(H, H)=10.1 Hz, 1H, Ph-GH₂); 1.59 (m, 4H, N—CH₂-CH₂); 1.43 (m, 2H, N—CH₂—CH₂—CH₂); 0.91 (d, ³J(H, H)=6.5 Hz, 3H, CH₃). ¹³C NMR (CDCl₃, 100.6 MHz, 25° C., δ=ppm): 141.0, 129.2, 128.1, 125.7, 62.1, 49.6, 39.1, 26.4, 24.9, 14.2. GC-MS: m/z=203 [M⁺], 112 [M⁺—C₆H₅—CH₂], 91, 69. MS (Cl, isobutane): 204 [M⁺+H], 112 [M⁺—C₆H₅—CH₂], 86. Anal. calc. for C₁₄H₂₁N: C 82.70, H 10.41, N 6.89. found: C 82.68, H 10.42, N 6.85.

### Example 2

N-2-(1-Phenyl)propylmorpholine. Morpholine (2.5 mmol; 218 μl) and allylbenzene (5 mmol; 662 μ) are reacted at 50° C. according to the general working procedure with addition of 20 mol % of n-BuLi solution (0.5 mmol; 313 μl). The product is purified by column chromatography (n-hexane/ethyl acetate=1:1). N-2-(1-Phenyl)-propylmorpholine is obtained as a colorless oil. Yield: 88% (GC); 80% (isolated). ¹H NMR (CDCl₃, 400.1 MHz, 25° C., δ=ppm): 7.26 (m, 2H, phenyl); 7.16 (m, 3H, phenyl); 3.72 (m, 4H, O—CH₂); 2.99 (dd, ²J(H, H)=13.1 Hz, ³J(H, H)=4.4 Hz, 1H, Ph-CH₂); 2.75 (ddq, ³J(H, H)=9.7 Hz, ³J(H, H)=6.5 Hz, ³J(H, H)=4.4 Hz, 1H, Ph-CH₂-Ch); 2.60 (m, 4H, N—CH₂); 2.39 (dd, ²J(H, H)=13.1 Hz, ³J(H, H)=9.7 Hz, 1H, Ph—OH₂); 0.94 (d, ³J(H, H)=6.5 Hz, 3H, CH₃). ¹³C NMR (CDCl₃, 100.6 MHz, 25° C., δ=ppm): 140.3, 129.2, 128.2, 125.8, 67.3, 61.6, 49.0, 39.1, 14.2. GC-MS: m/z=114 [M⁺—C₆H₅—CH₂], 91, 70. MS (Cl, isobutane): 206 [M⁺+H], 114 [M⁺—C₆H₅—CH₂]. Anal. calc. for C₁₃H₁₉NO: C 76.06, H 9.33, N 6.82. found: C 76.06, H 9.19, N 6.88.

### Example 3

N-2-(1-Phenyl)propyl-N-benzylamine. Benzylamine (2.5 mmol; 273 μl) and allylbenzene (5 mmol; 662 μl) are reacted at 50° C. according to the general working procedure with addition of 20 mol % of n-BuLi solution (0.5 mmol; 313 μl). The product is purified by column chromatography (n-hexane/ethyl acetate=4:1). N-2-(1-Phenyl)propyl-N-benzylamine is obtained as a colorless oil. Yield: 65% (GC); 60% (isolated). ¹H NMR (CDCl₃, 400.1 MHz, 25° C., δ=ppm): 7.29-7.12 (m, 10H, Phenyl); 3.83 (d, ²J(H, H)=13.3 Hz, 1H, Ph—CH₂—N); 3.72 (d, ²J(H, H)=13.3 Hz, 1H, Ph-CH₂—N); 2.92 (sext, 1H, ³J(H, H)=³J(H, H)=6.3 Hz, 1H, Ph—OH₂—OH); 2.76 (dd, ²J(H, H)=13.3 Hz, ³J(H, H)=6.3 Hz, 1H, Ph—CH₂—CH); 2.62 (dd, ²J(H, H)=13.3 Hz, ³J(H, H)=6.3 Hz, 1H, Ph—CH₂—CH); 1.54 (s, 1H, NH); 1.08 (d, ³J(H, H)=6.3 Hz, 3H, OH₃). ¹³C NMR (CDCl₃, 100.6 MHz, 25° C., δ=ppm): 140.4, 139.4, 129.3, 128.3, 128.3, 127.9, 126.8, 126.1, 53.7, 51.2, 43.5, 20.1. GC-MS: m/z=134 [M⁺—C₆H₅-CH₂], 91, 65. MS (Cl, isobutane): 226 [M⁺+H], 134 [M⁺—C₆H₅—CH₂]. Anal. calc. for C₁₆H₁₉N: C 85.28, H 8.50, N 6.22. found: C 85.19, H 8.51, N 6.19.

### Example 4

N—(S)-1-Phenylethyl-N-2-(1-phenyl)propylamine. S—(−)-α-Methylbenzylamine (2.5 mmol; 318 μl) and allylbenzene (5 mmol; 662 μl) are reacted at 50° C. according to the general working procedure with addition of 20 mol % of n-BuLi solution (0.5 mmol; 313 μl) and 20 mol % of tetramethylethylenediamine (0.5 mmol, 75 μl). The product is purified by column chromatography (n-hexane/ethyl acetate=2:1). N—(S)-1-Phenylethyl-N-2-(1-phenyl)propylamine is obtained as a colorless oil. Yield: 32% (GC); 30% (isolated).—¹H NMR (CDCl₃, 400.1 MHz, 25° C., δ=ppm): 7.36-6.94 (m, 2. 10H, phenyl); 3.93, 3.88 (2. q, ³J(H, H)=6.6 Hz, 2. 1H, Ph—CHMe—N); 2.88, 2.66 (2. dd, ²J(H, H)=12.9 Hz, ³J(H, H)=5.0 Hz, 2. 1H, Ph—CH₂); 2.77, 2.65 (2. m, 2. 1H, Ph—CH₂—CH; 2.59, 2.50 (2. dd, ²J(H, H)=12.9 Hz, ³J(H, H)=7.5 Hz, 2. 1H, Ph—CH₂); 1.46 (2. s, 2. 1H. N; 1.31, 1.27 (2. d, ³J(H, H)=6.7 Hz, 2. 3H, Ph—CH(CH₃)—N); 1.05, 0.92 (2. d, ³J(H, H)=6.3 Hz, 2. 3H, Ph—CH₂—CH—CH₃). ¹³C NMR (CDCl₃, 100.6 MHz, 25° C., δ=ppm): 146.1, 145.4, 139.5, 139.3, 129.4, 129.2, 128.4, 128.3, 128.3, 128.2, 126.8, 126.6, 126.5, 126.3, 126.1, 125.9, 55.3, 54.8, 51.9, 50.8, 44.2, 42.5, 25.0, 24.5, 21.1, 19.9. GC-MS: m/z=239 [M⁺], 148 [M⁺—C₆H₅—CH₂], 105 [C₆H₅—CH—CH₃], 91, 79. Anal. calc. for C₁₇H₂₁N: C 85.30, H 8.84, N 5.85. found: C 85.20, H 8.88, N 5.86.

### Example 5

N-n-Butyl-N-2-(1-phenyl)propylamine. n-Butylamine (2.5 mmol; 247 μl) and allylbenzene (5 mmol; 662 μl) are reacted at 50° C. according to the general working procedure with addition of 20 mol % of n-BuLi solution (0.5 mmol; 313 μl ). The product is purified by column chromatography (n-hexane/ethyl acetate=1:2). N-n-Butyl-N-2-(1-phenyl)propylamine is obtained as a colorless oil. Yield: 62% (GC); 54% (isolated). —¹H NMR (CDCl₃, 400.1 MHz, 25° C., δ=ppm): 7.27 (m, 2H, phenyl); 7.17 (m, 3H, phenyl); 2.87 (sext, ³J(H, H)=³J(H, H)=6.5 Hz, 1H, Ph—CH₂—); 2.73 (dd, ²J(H, H)=13.3 Hz, ³J(H, H)=6.5 Hz, 1H, Ph—CH₂); 2.65 (ddd, ²J(H, H)=11.1 Hz, ³J(H, H)=8.1 Hz, ³J(H, H)=6.5 Hz, 1H, N—CH₂); 2.58 (dd, ²J(H, H)=13.3 Hz, ³J(H, H)=6.5 Hz, 1H, Ph—CH₂); 2.51 (ddd, ²J(H, H)=11.1 Hz, ³J(H, H)=7.6 Hz, ³J(H, H)=6.7 Hz, 1H, N—CH₂); 1.40 (m, 2H, N—CH₂—CH₂); 1.37 (s, 1H, NM; 1.25 (sext, ³J(H, H)=³J(H, H)=7.4 Hz, 2H, CH₂—CH₂—CH₃); 1.04 (d, ³J(H, H)=6.5 Hz, 3H, CH—CH₃); 0.85 (t, ³J(H, H)=7.4 Hz, 3H, CH₂—CH₃). ¹³C NMR (CDCl₃, 100.6 MHz, 25° C., δ=ppm): 139.5, 129.2, 128.3, 126.1, 54.7, 47.0, 43.6, 32.3, 20.4, 20.2, 13.9. GC-MS: m/z=191 [M⁺], 176 [M⁺—CH₃], 100 [M⁺—C₆H₅—CH₂], 91, 77. Anal. calc. for C₁₃H₂₁N: C 81.61, H 11.06, N 7.32. found: C 81.44, H 10.99, N 7.20.

### Example 6

N-n-Butyl-N-methyl-N-2-(1-phenyl)propylamine. n-Butylmethylamine (2.5 mmol; 296 μl) and allylbenzene (5 mmol; 662 μl) are reacted at room temperature according to the general working procedure with addition of 20 mol % of n-BuLi solution (0.5 mmol; 313 μl). The product is purified by column chromatography (n-hexane/ethyl acetate=3:1). N-n-Butyl-N-methyl-N-2-(1-phenyl)propylamine is obtained as a colorless oil. Yield: 66% (GC); 61% (isolated).—¹H NMR (CDCl₃, 400.1 MHz, 25° C., δ=ppm): 7.26 (m, 2H, phenyl); 7.17 (m, 3H, phenyl); 2.94 (dd, ²J(H, H)=12.5 Hz, ³J(H, H)=4.0 Hz, 1H, Ph—CH₂); 2.89 (m, 1H, Ph—CH₂—Ch); 2.44 (m, 2H, N—CH₂); 2.38 (dd, ²J(H, H)=12.5 Hz, ³J(H, H)=9.5 Hz, 1H, Ph—CH₂); 2.28 (s, 3H, N—CH₃); 1.46 (m, 2H, N—CH₂—CH₂); 1.31 (m, 2H, CH₂—CH₂—CH₃); 0.91 (t, ³J(H, H)=7.3 Hz, 3H, CH₂—CH₃); 0.90 (d, ³J(H, H)=6.9 Hz, 3H, CH—CH₃). ¹³C NMR (CDCl₃, 100.6 MHz, 25° C., (=ppm): 140.8, 129.2, 128.2, 125.7, 60.3, 53.2, 39.0, 37.1, 30.4, 20.7, 14.1, 13.9. GC-MS: m/z=205 [M⁺], 190 [M⁺—CH₃ ], 162 [M⁺—CH₂CH₂CH₃], 114 [M⁺—C₆H₅—CH₂], 91, 72, 58. Anal. calc. for C₁₄H₂₃N: C 81.89, H 11.29, N 6.82. found: C 81.31, H 11.19, N 6.76.

### Example 7

N-2-(1-Phenyl)propylaniline. Aniline (2.5 mmol; 228 μl) and allylbenzene (2.5 mmol; 331 μl) are reacted at 120° C. according to the general working procedure with addition of 30 mol % of n-BuLi solution (0.75 mmol; 469 μl) and 30 mol % of KO^{t}Bu (0.75 mmol, 84 mg). The product is purified by column chromatography (n-hexane/ethyl acetate=15:1). N-2-(1-Phenyl)propylaniline is obtained as a colorless oil. Yield: 54% (GC); 50% (isolated).—¹H NMR (CDCl₃, 400.1 MHz, 25° C., δ=ppm): 7.28 (m, 2H, phenyl); 7.18 (m, 3H, phenyl); 7.16 (m, 2H, phenyl); 6.68 (m, 1H, phenyl); 6.62 (m, 2H, phenyl); 3.75 (m, 1H, Ph—CH₂—Ch); 3.61 (s, 1H, NM; 2.93 (dd, ²J(H, H)=13.4 Hz, ³J(H, H)=4.8 Hz, 1H, Ph—CH₂); 2.68 (dd, ²J(H, H)=13.4 Hz, ³J(H, H)=7.3 Hz, 1H, Ph—CH₂); 1.13 (d, ³J(H, H)=6.3 Hz, 3H, CH₃). ¹³C NMR (CDCl₃, 100.6 MHz, 25° C., δ=ppm): 147.1, 138.5, 129.5, 129.3, 128.3, 126.2, 117.2, 113.4, 49.4, 42.2, 20.1. GC-MS: m/z=211 [M⁺], 120 [M⁺—C₆H₅—CH₂], 91, 77. Anal. calc. for C₁₅H₁₇N: C 85.26, H 8.11, N 6.63. found: C 85.41, H 8.10, N 6.64.

### Example 8

N-2-(1-Phenyl)butylpiperidine. Piperidine (2.5 mmol; 247 μl) and 4-phenyl-1-butene (5 mmol; 746 μl) are reacted at 50° C. according to the general working procedure with addition of 20 mol % of n-BuLi solution (0.5 mmol; 313 μl). The product is purified by column chromatography (n-hexane/ethyl acetate=4:1). N-2-(1-Phenyl)-butylpiperidine is obtained as a colorless oil. Yield: 59% (GC); 57% (isolated).—¹H NMR (CDCl₃, 400.1 MHz, 25° C., δ=ppm): 7.25 (m, 2H, phenyl); 7.16 (m, 3H, phenyl); 2.94 (dd, ²J(H, H)=12.9 Hz, ³J(H, H)=4.0 Hz, 1H, Ph—CH₂); 2.66-2.40 (m, 5H, N—CH₂, Ph—CH₂—CH); 2.33 (dd, ²J(H, H)=12.9 Hz, ³J(H, H)=9.0 Hz, 1H, Ph—CH₂); 1.54 (m, 4H, N—CH₂—CH₂); 1.49-1.38 (m, 3H, N—CH₂—CH₂—CH₂, Ph—CH₂—CH—CH₂); 1.32 (m, 1H, Ph—CH₂—CH—CH₂); 0.81 (t, ³J(H, H)=7.3 Hz, 3H, CH₃). ¹³C NMR (CDCl₃, 100.6 MHz, 25° C., δ=ppm): 141.7, 129.2, 128.1, 125.4, 68.6, 49.6, 35.5, 26.6, 25.1, 23.3, 11.7. GC-MS: m/z=217 [M⁺], 188 [M⁺—CH₂CH₃], 126 [M⁺—C₆H₅—CH₂], 91, 69. Anal. calc. for C₁₅H₂₃N: C 82.89, H 10.67, N 6.44. found: C 82.80, H 10.62, N 6.33.

1. A process for the preparation of 2-arylethylamine derivatives of the formula (I),
aryl-CHR¹—CR²(NR³R⁴)—(CHR⁵R⁶)ₙ—CHR⁷R⁸ (I)
in which R¹ to R⁶
can be, identically or differently, hydrogen, C₁-C₂₄-alkyl, C₃-C₁₂-cycloalkyl, where the cycle can also contain 1-2 heteroatoms selected from the group consisting of N, O, S, P and the ring size is 3-12, phenyl, naphthyl, fluorenyl, C₆-C₁₄-aryl, C₂-C₁₃-heteroaryl, where the number of heteroatoms selected from the group consisting of N, O, S, P can be 1 to 4 and the ring size is 3-14, where these groups themselves can in each case be mono- or polysubstituted and these substituents in this case independently of one another contain hydrogen, C₁-C₂₀-alkyl, C₁-C₁₀-fluoroalkyl, C₃-C₁₂-cycloalkyl, where the cycle can also contain 1-2 heteroatoms selected from the group consisting of N, O, S, P and the ring size is 3-12, phenyl, C₆-C₁₄-aryl, C₃-C₁₃-heteroaryl, where the number of heteroatoms selected from the group consisting of N, O, S, P can be 1 to 4 and the ring size is 3-14, C₁-C₁₀-alkoxy, C₆-C₁₄-aryloxy, C₃-C₁₃-heteroaryloxy, where the number of heteroatoms selected from the group consisting of N, O, S, P can be 1 to 4 and the ring size is 3-14, C₁-C₉-trifluoromethylalkyl, trifluoromethyl, fluoro, nitro, hydroxy, trifluoromethylsulfonato, thio, thiolato, C₁-C₈-substituted amino of the forms NH-alkyl-C₁-C₈, NH-aryl-C₅-C₆, N-alkyl₂-C₁-C₈, N-aryl₂-C₅-C₆, NHCOH, NH—CO-alkyl-C₁-C₈, NH—CO-aryl-C₅-C₆, NHCOO-alkyl-(C₁-C₄); NHCOO-aryl-(C₃-C₈); cyano, C₁-C₆-acyloxy, C₁-C₇-CO₂H, SO₂-aryl-(C₃-C₆), SO-aryl-(C₃-C₆), SO₂-alkyl-(C₁-C₆), SO-alkyl-(C₁-C₆), sulfinato, sulfonato of the forms SO₃H and SO₃R⁹, P(phenyl)₂, P-alkyl₂-(C₁-C₈), P-aryl₂-(C₃-C₈), PO-phenyl₂, POalkyl₂-(C₁-C₄), phosphato of the forms PO₃H₂, PO₃HR⁹, PO₃ R⁹₂, CONH₂, CONR⁹₂, CONHR⁹, where R⁹ is either a C₁-C₈-alkyl or C₆-aryl, C₁-C₆-trialkylsilyl, where also the individual substituents R⁵ and R⁶ in the n subunits can be different substituents, and
in which R⁷ and R⁸
can be, identically or differently, hydrogen, C₁-C₂₄-alkyl, C₃-C₁₂-cycloalkyl, where the cycle can also contain 1-2 heteroatoms selected from the group consisting of N, O, S, P and the ring size is 3-12, phenyl, naphthyl, fluorenyl, C₆-C₁₄-aryl, C₂-C₁₃-heteroaryl, where the number of heteroatoms selected from the group consisting of N, O, S, P can be 1 to 4 and the ring size is 3-14, fluorine, OH, NO₂, CN, O—C₁-C₂₄-alkyl, O—C₃-C₁₂-cycloalkyl, where the cycle can also contain 1-2 heteroatoms selected from the group consisting of N, O, S, P and the ring size is 3-12,
O—C₆-C₁₄-aryl, O—C₂-CO₃-heteroaryl, where the number of heteroatoms selected from the group consisting of N, O, S, P can be 1 to 4 and the ring size is 3-14, N—(C₁-C₂₄)₂-alkyl, N—(C₃-C₈)₂-cycloalkyl, where the cycle can also contain 1-2 heteroatoms selected from the group consisting of N, O, S, P and the ring size is 3-12,
N—(C₆-C₁₄-aryl)₂, N—(C₂-C₁₃-heteroaryl)₂, where the number of heteroatoms selected from the group consisting of N, O, S, P can be 1 to 4 and the ring size is 3-14,
O₂C—C₁-C₂₄-alkyl, O₂C—C₃-C₈-cycloalkyl where the cycle can also contain 1-2 heteroatoms selected from the group consisting of N, O, S, P and the ring size is 3-12, O₂C—C₆-C₁₄-aryl,
NR¹⁰—CO—(C₁-C₂₄)₂-alkyl, NR¹⁰—CO—(C₃-C₁₂)₂-cycloalkyl, where the cycle can also contain 1-2 heteroatoms selected from the group consisting of N, O, S, P and the ring size is 3-12,
NR¹⁰—CO—(C₆-C₁₄-aryl)₂, NR¹⁰—CO—(C₂-C₁₃-heteroaryl)₂, where the number of heteroatoms selected from the group consisting of N, O, S, P can be 1 to 4 and the ring size is 3-14, where R¹⁰ is C₁-C₈-alkyl or C₆-aryl
Si-(C₁-C₂₄)₄-alkyl, Si—(C₃-C₁₂)₄-cycloalkyl, where the cycle can also contain 1-2 heteroatoms selected from the group consisting of N, O, S, P and the ring size is 3-12,
Si—(C₆-C₁₄-aryl)₄, Si—(C₂-C₁₃-heteroaryl)₄, where the number of heteroatoms selected from the group consisting of N, O, S, P can be 1 to 4 and the ring size is preferably 3-14,
where these groups themselves can in each case be mono- or polysubstituted and these substituents in this case independently of one another can be hydrogen, C₁-C₂₀-alkyl, C₁-C₁₀-fluoroalkyl, C₃-C₁₂-cycloalkyl, where the cycle can also contain 1-2 heteroatoms selected from the group consisting of N, O, S, P and the ring size is 3-12, phenyl, C₆-C₁₄-aryl, C₃-C₁₃-heteroaryl, where the number of heteroatoms selected from the group consisting of N, O, S, P can be 1 to 4 and the ring size is 3-14, C₁-C₁₀-alkoxy, C₆-C₁₄-aryloxy, C₃-C₁₃-heteroaryloxy, where the number of heteroatoms selected from the group consisting of N, O, S, P can be 1 to 4 and the ring size is 3-14, C₁-C₉-trifluoromethylalkyl, trifluoromethyl, fluoro, nitro, hydroxyl, trifluoromethylsulfonato, thio, thiolato, C₁-C₈-substituted amino of the forms NH-alkyl-C₁-C₈, NH-aryl-C₅-C₆, N-alkyl₂-C₁-C₈, N-aryl₂-C₅-C₆, NHCOH, NH—COalkyl-C₁-C₈, NH—CO-aryl-C₅-C₆, NHCOO-alkyl-(C₁-C₄); NHCOO-aryl-(C₃-C₈); cyano, C₁-C₆-acyloxy, C₁-C₇-CO₂H, SO₂-aryl-(C₃-C₆), SO-aryl-(C₃-C₆), SO₂-alkyl-(C₁-C₆), SO-alkyl-(C₁-C₆), sulfinato, sulfonato of the forms SO₃H and SO₃R⁹, P(phenyl)₂, P-alkyl₂-(C₁-C₈), P-aryl₂-(C₃-C₈), PO-phenyl₂, POalkyl₂(C₁-C₄), phosphato of the forms PO₃H₂, PO₃HR⁹, PO₃R⁹₂, CONH₂, CONR⁹₂, CONHR⁹, where R⁹ is either a C₁-C₈-alkyl or C₆-aryl, C₁-C₆-trialkylsilyl,
and in which aryl in formula (I)
can be C₃-C₁₄-aryl, C₂-C₁₃-heteroaryl, where the number of heteroatoms selected from the group consisting of N, O, S, P can be 1 to 4 and the ring size is 3-14,
where these groups themselves can in each case be mono- or polysubstituted and these substituents in this case independently of one another can be hydrogen, C₁-C₂₀-alkyl, C₁-C₁₀-fluoroalkyl, C₃-C₁₂-cycloalkyl, where the cycle can also contain 1-2 heteroatoms selected from the group consisting of N, O, S, P and the ring size is 3-12, phenyl, C₆-C₁₄-aryl, C₂-C₁₃-heteroaryl, where the number of heteroatoms selected from the group consisting of N, O, S, P can be 1 to 4 and the ring size is 3-14, C₁-C₁₀-alkoxy, C₆-C₁₄-aryloxy, C₁-C₉-trifluoromethylalkyl, trifluoromethyl, fluoro, nitro, hydroxyl, trifluoromethylsulfonato, thio, thiolato, C₁-C₈-substituted amino of the forms NH-alkyl-C₁-C₈, NH-aryl-C₅-C₆, N-alkyl₂-C₁-C₈, N-aryl₂-C₅-C₆, N-alkyl₃-C₁-C₈+, N-aryl₃-C₅-C₆+, NHCOH, NH—COalkyl-C₁-C8, NH—CO-aryl-C₅-C₆, NHCOO-alkyl-(C₁-C₄); NHCOO-aryl-(C₃-C₈); cyano, C₁-C₆-acyloxy, SO₂-aryl-(C₃-C₆), SO-aryl-(C₃-C₆) SO₂-alkyl-(C₁-C₆), SO-alkyl-(C₁-C₆), sulfinato, sulfonato of the forms SO₃H and SO₃R⁹, P(phenyl)₂, CHCHCO₂H, P-alkyl₂-(C₁-C₈), P-aryl₂-(C₃-C₈), PO-phenyl₂, POalkyl₂-(C₁-C₄), phosphato of the forms PO₃H₂, PO₃HR⁹ and PO₃R⁹₂, CONH₂, CONR⁹₂, CONHR⁹ where R⁹ is either C₁-C₈-alkyl or C₆-Aryl, C₁-C₆-trialkylsilyl, and
in which n is a number between 0 and 11,
by reaction of aromatic olefins of the general formulae (IIa-c)
aryl-CHR¹—CHR²—(CR⁵R⁶)ₐ—CR⁵═CR⁶—(CR⁵R⁶)_{b}—CHR⁷R⁸ (IIa)
aryl-CHR¹—CR²═CR⁵—(CR⁵R⁶)_{c}—CHR⁷R⁸ (IIb)
aryl-CHR¹—CHR²—(CR⁵CR⁶)_{c}—CR⁵═CR⁷CR⁸ (IIc)
with amines of the general formula (III),
R³R⁴NH (III)
in which in the formulae of the type II and III R¹ to R⁸ have the meaning indicated beforehand for the formulae of the type (I) and a and b are a number between 0-9 with the condition that a+b is <10 and c is a number between 0-10,
in the presence of a base.

2. The process as claimed in claim 1, wherein the substituents R¹ to R⁶ are hydrogen, C₁-C₈-alkyl, C₆-C₁₄-aryl, C₃-C₁₂-cycloalkyl, where the cycle can also contain 1-2 heteroatoms selected from the group consisting of N, O, S, P and the ring size is 3-12,
where these groups themselves can in each case be mono- to trisubstituted and these substituents in this case independently of one another can be hydrogen, C₁-C₂₀-alkyl, C₆-C₁₄-aryl, C₃-C₁₂-cycloalkyl, where the cycle can also contain 1-2 heteroatoms selected from the group consisting of N, O, S, P and the ring size is 3-12, C₁-C₁₀-alkoxy, C₁-C₁₄-aryloxy, C₃-C₁₃-heteroaryloxy, where the number of heteroatoms selected from the group consisting of N, O, S, P can be 1 to 4 and the ring size is 3-14, fluoro, trifluoromethyl, N-alkyl₂-C₁-C₈, N-aryl₂-C₅-C₆.

3. The process as claimed in one of claims 1 and 2, wherein the substituents R¹ to R⁶ independently of one another are hydrogen, C₁-C₈-alkyl, C₆-C₁₄-aryl, C₃-C₁₂-cycloalkyl, where the cycle can also contain 1-2 heteroatoms selected from the group consisting of N, O, S, P and the ring size is 3-12.

4. The process as claimed in one of claims 1 to 3, wherein the substituents R⁷ and R⁸ independently of one another can be hydrogen, fluoro, trifluoromethyl, C₆-C₁₄-aryl, O—C₁-C₈-alkyl, O—C₃-C₁₂-cycloalkyl, where the cycle can also contain 1-2 heteroatoms selected from the group consisting of N, O, S, P and the ring size is 3-12, O—C₆-C₁₄-aryl, O—C₂-C₁₃-heteroaryl, where the number of heteroatoms selected from the group consisting of N, O, S, P can be 1 to 4 and the ring size is preferably 3-14,
N—(C₁-C₈)₂-alkyl, N—(C₃-C₈)₂-cycloalkyl, where the cycle can also contain 1-2 heteroatoms selected from the group consisting of N, O, S, P and the ring size is 3-12,
N—(C₆-C₁₄-aryl)₂, N—(C₂-C₁₃-heteroaryl)₂, where the number of heteroatoms selected from the group consisting of N, O, S, P can be 1 to 4 and the ring size is 3-14,
where these groups themselves in each case can be mono- to trisubstituted and these substituents in this case independently of one another can be hydrogen, C₁-C₂₀-alkyl, C₃-C₁₂-cycloalkyl, where the cycle can also contain 1-2 heteroatoms selected from the group consisting of N, O, S, P and the ring size is 3-12, phenyl, C₆-C₁₄-aryl, C₃-C₁₃-heteroaryl, where the number of heteroatoms selected from the group consisting of N, O, S, P can be 1 to 4 and the ring size is 3-14, C₁-C₁₀-alkoxy, C₆-C₁₄-aryloxy, C₃-C₁₃-heteroaryloxy, where the number of heteroatoms selected from the group consisting of N, O, S, P can be 1 to 4 and the ring size is 3-14, N-alkyl₂-C₁-C₈, N-aryl₂-C₅-C₆, trifluoromethyl, fluoro.

5. The process as claimed in one of claims 1 to 4, wherein the substituents R⁷ and R⁸ independently of one another are hydrogen, C₃-C₁₄-aryl, C₂-C₁₃heteroaryl, where the number of heteroatoms selected from the group consisting of N, O, S, P can be 1 to 4 and the ring size is 3-14.

6. The process as claimed in one of claims 1 to 5, wherein the aryl radical in formula (I) is C₃-C₁₄-aryl, C₂-C₁₃-heteroaryl, where the number of heteroatoms selected from the group consisting of N, O, S, P can be 1 to 4 and the ring size is 3-14, where these groups themselves can in each case be mono- to trisubstituted and these substituents in this case independently of one another are hydrogen, C₁-C₂₀-alkyl, C₃-C₁₂-cycloalkyl, where the cycle can also contain 1-2 heteroatoms selected from the group consisting of N, O, S, P and the ring size is 3-12, phenyl, C₆-C₁₄-aryl, C₂-C₁₃-heteroaryl, where the number of heteroatoms selected from the group consisting of N, O, S, P can be 1 to 4 and the ring size is 3-14, C₁-C₁₀-alkoxy, C₁-C₉-trifluoromethylalkyl, trifluoromethyl, fluoro, nitro, hydroxyl, trifluoromethylsulfonato, thio, thiolato.

7. The process as claimed in one of claims 1 to 6, wherein a mixture of starting materials of the formula (II) is employed.

8. The process as claimed in one of claims 1 to 7, wherein the base employed is a Brönsted base.

9. The process as claimed in claims 1 to 8, wherein the base employed is an alkali metal and/or alkaline earth metal alkoxide, alkali metal and/or alkaline earth metal amide, alkali metal and/or alkaline earth metal element and/or alkali metal and/or alkaline earth metal hydrocarbon and/or hydroxide, preferably of lithium, sodium, potassium, calcium, magnesium, cesium and/or superbases, such as alkali metal-doped zeolites.

10. The process as claimed in one of claims 1 to 9, wherein the base is used in catalytic amounts from 0.01 eq to 0.5 eq (based on amine).

11. The process as claimed in one of claims 1 to 10, wherein the reaction is carried out in an inert organic solvent or solvent mixture.

12. The process as claimed in one of claims 1 to 10, wherein no solvent is used.

13. The process as claimed in one of the above claims, wherein the reaction proceeds at temperatures from −70 to 200° C.; preferably at 0 to 180° C., particularly preferably 20 to 140° C.

14. The process as claimed in one of the above claims, wherein the reaction is carried out in the presence of a chelating amine as cocatalyst.
